(19) United States
(12) Patent Application Publication
(10) Pub. No.: US 2014/0303493 A1
(43) Pub. Date: Oct. 9, 2014

(54) SURGERY ASSISTING APPARATUS

(71) Applicants: Kabushiki Kaisha Toshiba, Minato-ku (JP); Toshiba Medical Systems Corporation, Otawara-Shi (JP)

(72) Inventors: Tomohiro KAWASAKI, Otawara-Shi (JP); Takuma IGARASHI, Nasushiobara-Shi (JP); Megumu FUJIWARA, Sakura-Shi (JP)

(73) Assignees: Kabushiki Kaisha Toshiba, Minato-ku (JP); Toshiba Medical Systems Corporation, Otawara-Shi (JP)

(21) Appl. No.: 14/312,167

(22) Filed: Jun. 23, 2014

Related U.S. Application Data
(63) Continuation of application No. PCT/JP2013/080205, filed on Nov. 8, 2013.

(30) Foreign Application Priority Data
Nov. 15, 2012 (JP) 2012-251041

(51) Int. Cl. A61F 2/46; A61B 6/00; A61B 6/03; A61F 2/38; A61F 2/28; A61B 6/12; A61F 2/32

(52) U.S. Cl. CPC A61F 2/4657; A61B 6/12; A61B 6/466; A61B 6/5205; A61B 6/504; A61B 6/506; A61B 6/505; A61F 2/32; A61F 2/38; A61F 2/28; A61B 6/032; A61F 2002/4663; A61F 2002/2825
USPC 600/424

(57) ABSTRACT

A surgery assisting apparatus of an embodiment is characterized by including a bone object extracting section configured to produce a 3D object image in which first and second bone objects are separated and extracted from a 3D image in which a diseased part including a joint and first and second bone parts movably connected with each other via the joint, an object position aligning section configured, while extracting the first and second bone parts in an X-ray image in which the diseased part is intra-operatively photographed and producing an intra-operative X-ray bone part extracted image, to align the 3D object image in position in such a way that the first bone object and the second bone object agree with the first bone part and the second bone part in the X-ray bone part extracted image, respectively, so as to produce a reference image, and a display section on which the X-ray image and the reference image are displayed.

### CROSS-REFERENCE TO RELATED APPLICATIONS

This application is a Continuation application of No. PCT/JP2013/80205, filed on Nov. 8, 2013, and the PCT application is based upon and claims the benefit of priority from Japanese Patent Applications No. 2012-251041 filed on Nov. 15, 2012, the entire contents of which are incorporated herein by reference.

### FIELD

An embodiment of the present invention relates to a surgery assisting apparatus.

### BACKGROUND

A method for shaping an artificial hip joint by removing a face damaged by osteonecrosis of the femoral head, etc., to replace that with an artificial hip joint is known as a cure for a disease of a hip joint such as hip osteoarthritis, rheumatism and so on. According to an ordinary method for artificial hip joint shaping, resect the femoral head, and then implant four implant parts called a stem, a thigh bone head, a liner and an acetabular cup into a hip joint part of a patient.

As conditions of the hip joint differ patient by patient, it is important that the implant parts be suitably selected correspondingly to the patient and that where to insert the implant parts be suitably decided correspondingly to the patient. Thus, it is practiced to establish a surgery plan including sizes of the implant parts being three-dimensional optimums or where to insert the implant parts by the use of a CT image of the relevant patient, e.g., as disclosed in Japanese Unexamined Patent Publication No. 2006-263241, etc.

Further, it is often practiced, as well, to capture an image of a patient intra-operatively by means of an X-ray imaging apparatus and to conduct a surgery while monitoring an obtained X-ray image and confirming where to insert the implant parts at times.

A CT image used in a surgery plan is ordinarily photographed in condition that a patient is in a supine (face-up) posture and that the knees and the femurs of the patient are stretched straight. Meanwhile, as a hip joint replacement surgery is conducted in condition that a patient is in a lateral recumbent posture, the knees and the femurs are in bending conditions. Thus, the X-ray image intra-operatively photographed and the CT image photographed in advance are different from each other in how the joint requiring surgery bends. Thus, even if an attempt is intra-operatively made to compare the two images with each other, it is hardly known how the joint and the implant parts relate to one another in both the images.

Further, as the patient undergoing the surgery is in the lateral recumbent posture, the doctor's line of sight along which the patient undergoing the surgery is viewed is different from the direction in which the CT image in the surgery plan is photographed. Thus, even if an attempt is made to check whether intra-operative conditions in which the implant parts are inserted agree with conditions having been expected in time of the pre-surgery plan by referring to the CT image, it is hardly known how they relate to one another.

Meanwhile, it becomes more frequent in recent years to conduct a minimum invasive surgery (MIS), i.e., a surgery by invading through an extremely small resection area (e.g., resection area of 10 centimeters or below) from a viewpoint of reducing a burden on the patient, etc. As the resection area is small in this type of the minimum invasive surgery, it is hardly known intra-operatively how the thigh bone and the implant parts are relatively located to one another, and it is hardly known whether the implant parts are inserted into the right position in the right angle. Further, as the resection area is small, there is another problem in that it is hardly grasped intra-operatively how the blood vessels and the nerves not to be damaged run.

Thus, a surgery assisting apparatus which, while facilitating a comparison between how the implant parts are to be inserted in the pre-surgery plan phase and how the implant parts are inserted in the X-ray photographed image intra-operatively obtained, facilitates a comparison between intra-operative conditions of insertion of the implant parts and their surrounds as viewed by a surgeon from a small resection area, and conditions of insertion of the implant parts and their surrounds in the pre-surgery plan phase is demanded.

### SUMMARY

A surgery assisting apparatus of an embodiment is a surgery assisting apparatus configured to assist a surgery to replace a joint of a patient with an artificial joint, the surgery assisting apparatus includes a bone object extracting section configured to produce a 3D object image in which a first bone object and a second bone object are each separated and extracted from a 3D image in which a diseased part including the joint, a first bone part and a second bone part movably connected with the first bone part via the joint are photographed, the first bone object and the second bone object corresponding to the first bone part and the second bone part, respectively, an object position aligning section configured to input the X-ray image in which the diseased part is photographed in the course of the surgery of the patient, the object position aligning section being configured, while extracting the first bone part and the second bone part in the inputted X-ray image and producing an intra-operative X-ray bone part extracted image, to align the 3D object image in position in such away that the first bone object and the second bone object agree with the first bone part and the second bone part in the intra-operative X-ray bone part extracted image, respectively, so as to produce a reference image, and a display section on which the X-ray image and the reference image are displayed.

### BRIEF DESCRIPTION OF DRAWINGS

FIG. 1 depicts an exemplary setup of a surgery assisting apparatus 1 of a first embodiment;

FIGS. 2A to 2C schematically illustrate data processing to separate and extract bone objects from 3D image data;

FIGS. 3A to 3C schematically illustrate an intra-operative X-ray outline image;

FIGS. 4A to 4E schematically illustrate data processing to align positions of a CT object 3D image (before parts insertion) and an intra-operative X-ray outline image (before parts insertion) with each other;

FIGS. 5A to 5E schematically illustrate data processing to align positions of a CT object 3D image (after parts insertion) and an intra-operative X-ray outline image (after parts insertion) with each other;

FIGS. 6A to 6B depict another first exemplary image displayed on the display section;

FIG. 7 depicts another second exemplary image displayed on the display section;

FIG. 8 depicts an exemplary setup of a surgery assisting apparatus of a second embodiment;

FIG. 9 illustrates an exemplary display of a CT object 3D image as viewed along a surgeon's line of sight according to the second embodiment;

FIGS. 10A to 10B schematically illustrate data processing to a separate and extract a bone object and a blood vessel/nerve object from 3D image data according to a third embodiment;

FIGS. 11A to 11E schematically illustrate data processing to align positions of a CT object 3D image with a blood vessel/nerve object (before parts insertion) and an intra-operative X-ray outline image (before parts insertion) with each other;

FIGS. 12A to 12E schematically illustrates data processing to align positions of a CT object 3D image with a blood vessel/nerve object (after parts insertion) and an intra-operative X-ray outline image (after parts insertion) with each other;

FIG. 13 illustrates an exemplary display of a CT object 3D image with a blood vessel/nerve object as viewed along a surgeon's line of sight according to the second embodiment;

FIGS. 14A to 14F illustrate a first exemplary application of the surgery assisting apparatus to an artificial knee joint replacement surgery; and

FIGS. 15A to 15D illustrate a second exemplary application of the surgery assisting apparatus to an artificial knee joint replacement surgery.

### DESCRIPTION OF EMBODIMENTS

Embodiments of the invention will be explained below on the basis of the drawings.

A surgery assisting apparatus 1 of the embodiments is an apparatus which assists a surgery to replace a joint such as a hip joint, a knee joint, etc., with an artificial joint. An artificial hip joint replacement surgery will be explained below in brief for an explanation of the surgery assisting apparatus 1 taking the artificial hip joint replacement surgery as an example.

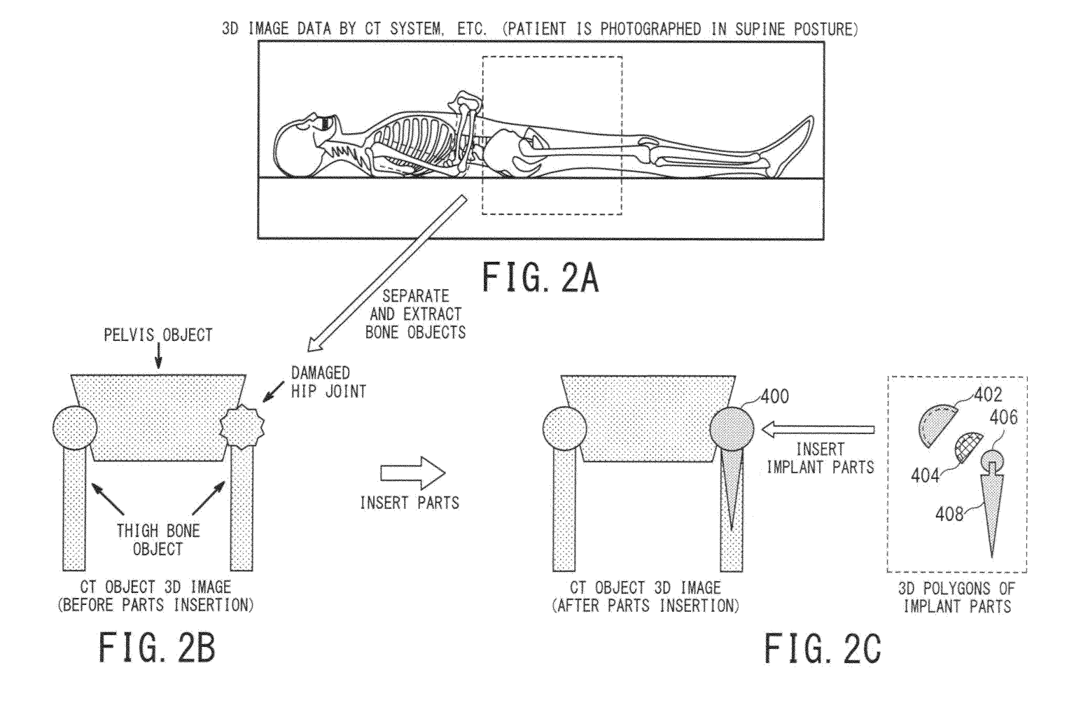

The artificial hip joint replacement surgery is a surgery to remove and replace a damaged face of a hip joint damaged by osteonecrosis of the femoral head, etc., with an artificial hip joint if a disease of the hip joint such as hip osteoarthritis, rheumatism and so on has worsened. The artificial hip joint is usually formed by four implant parts called a stem, a thigh bone head, a liner and an acetabular cup (see a drawing in a right portion of FIG. 2C). The artificial hip joint replacement surgery is conducted chiefly in a procedure shown below in condition that the patient is laid in a lateral recumbent posture in such a way that the joint requiring surgery comes to an upper side.

(1) Resect the skin, etc., so as to expose the hip joint being diseased. (2) Remove the femoral head (one of the head portions of the thigh bone closer to the hip joint). (3) Process the acetabular roof (a dented portion of the joint portion closer to the pelvis). (4) Insert the acetabular cup, one of the implant parts, into the processed dented portion of the joint portion closer to the pelvis, and then push the liner into there from the above. (5) Process the medullary cavity of the thigh bone. (6) Insert the stem, one of the implant parts, into the processed medullary cavity of the thigh bone. (7) Put the thighbone head to the head portion of the stem. (8) Fit the thigh bone head together with the acetabular cup via the liner, and then close the open wound.

The diseased portion is intra-operatively photographed in the above process, and a surgeon (doctor) checks where the implant parts (simply called the parts, hereafter) are inserted intra-operatively at any time by means of an intra-operative X-ray image obtained by X-ray photographing mentioned above.

Meanwhile, a CT 3D image of the diseased part of the patient photographed in advance by a CT apparatus is used in a preoperative plan, as described above. Further, it is practiced as well to extract bone parts of the pelvis and the thigh bone as bone objects from the CT 3D image, to insert part objects, i.e., the parts such as the stem, etc., modeled by 3D polygons, etc., for the extracted bone objects, and to decide the right parts selection and the right positions of parts insertion in advance in the preoperative plan.

As a patient being in a lateral recumbent posture is intra-operatively photographed by X-ray photographing, however, the intra-operative X-ray image is an image in which the knee joint is in bent condition. The CT 3D image used in the preoperative plan is usually an image in which a patient being in a supine posture is photographed, on the other hand, and thus is an image in which the hip joint and the knee are in stretched condition. Thus, even if an attempt is intra-operatively made to compare the intra-operative X-ray image and the CT 3D image obtained in the preoperative plan, there is a difference between the both in how the joint bends, and thus the CT 3D image obtained in the preoperative plan cannot be put to enough use.

Further, as the surgeon looks down at the patient being in the lateral recumbent posture, the surgeon's line of sight does not necessarily agree with a direction in which the CT 3D image obtained in the preoperative plan is displayed, and thus the CT 3D image obtained in the preoperative plan cannot be put to enough use from this viewpoint, either.

Further, in a case of a minimum invasive surgery conducted lots of times in recent years, there is a problem in that it is hardly grasped how the blood vessels and the nerves intra-operatively not to be damaged run. The surgery assisting apparatus 1 of the embodiments is to solve the problems described above.

### (1) First Embodiment

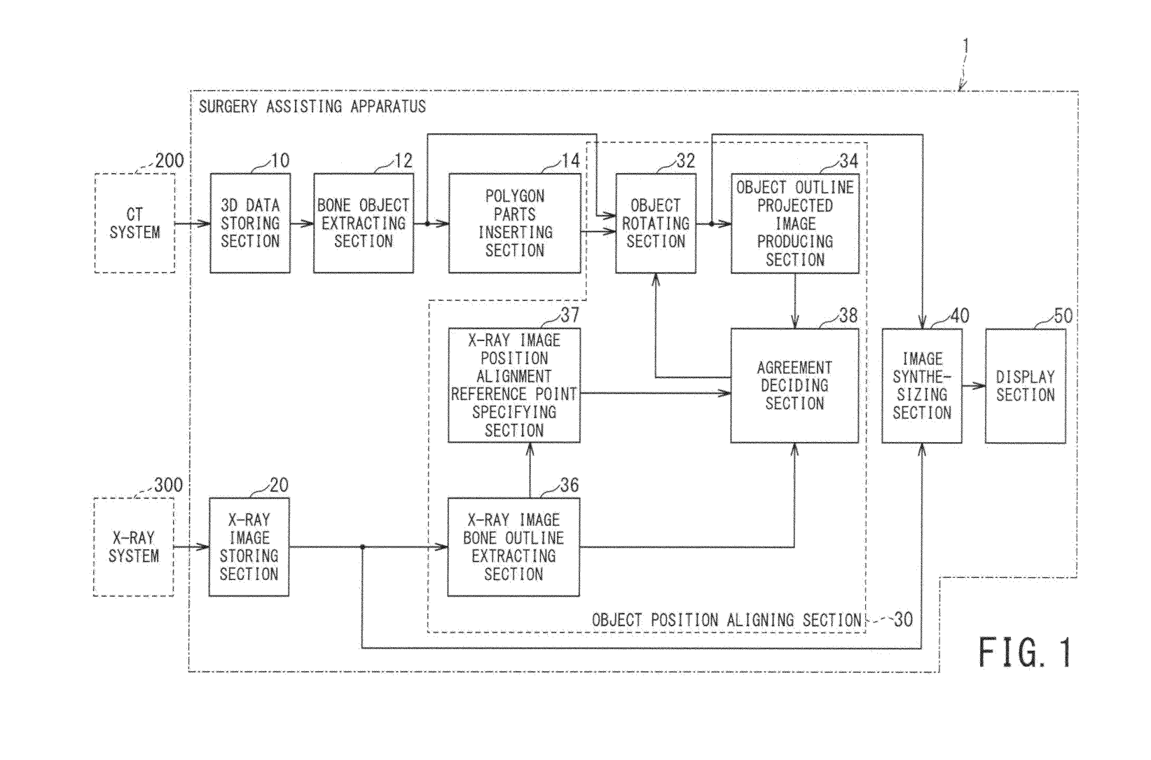

FIG. 1 depicts an exemplary setup of a surgery assisting apparatus 1 of a first embodiment.

The surgery assisting apparatus 1 of the embodiment is formed by having a 3D data storing section 10, a bone object extracting section 12, a polygon parts inserting section 14, an X-ray image storing section 20, an object position aligning section 30, an image synthesizing section 40, a display section 50, etc. Further, the object position aligning section 30 has, as its internal components, an object rotating section 32, an object outline projected image producing section 34, an X-ray image bone outline extracting section 36, an X-ray image position alignment reference point specifying section 37, an agreement deciding section 38, etc.

A versatile computer system, e.g., can be used as a basic hardware component for the surgery assisting apparatus 1 depicted in FIG. 1. Then, the components described above excepting the display section 50 can each be implemented by a program made run on a processor installed in the computer system. In this case, the program can be suitably stored in advance in a storage device in the computer system, or can be stored in a removable recoding medium such as a magnetic disk, a magneto-optical disk, an optical disk, a semiconductor memory, etc., and suitably installed into the computer system described above. Alternatively, the program can be installed into the computer system via a network connected to the computer system. Moreover, some or all of the respective components described above can be implemented by hardware devices such as logic circuits, ASIC, etc. Alternatively, the respective components described above can be implemented by hardware and software combinations.

3D image data preoperatively photographed by a CT system 200 is stored in the 3D data storing section 10 depicted in FIG. 1. Photographed areas of the 3D image data include the joint requiring surgery, the pelvis (first bone part) and the thigh bone (second bone part). Although it is assumed in an explanation below that the 3D image data is produced by the CT system 200, the 3D image data can be produced by an imaging system excepting the CT system 200, e.g., an MRI system.

The bone object extracting section 12 extracts 3D object data which corresponds to the pelvis, the right thigh bone and the left thigh bone (each called a bone object, hereafter) from the 3D image data stored in the 3D data storing section 10.

Specifically, extract an area of a CT value being 1000HU and over, e.g., as an entire area of the bone in the beginning. Then, perform image processing such as known dilation processing and erosion processing for a couple of voxels on the extracted entire area of the bone, so as to separate and extract bone objects which each corresponds to the pelvis and the left and right thigh bones (a pelvis object and left and right thigh bone objects).

FIGS. 2A and 2B schematically illustrate data processing to separate and extract the bone objects from the 3D image data. As depicted in FIG. 2A, the CT system 200 usually captures an image of a patient being in a supine posture, and obtains 3D image data. Thus, an image of the bone objects separated and extracted from the 3D image data is an image in which the hip joint is stretched and the left and right thigh bones are substantially parallel to each other as depicted in FIG. 2B (this image is called a “CT object 3D image (before parts insertion)”, hereafter).

The polygon parts inserting section 14 inserts implant parts 400 in a form of image data into a portion of the CT object 3D image which corresponds to the hip joint being the part requiring surgery, as depicted in FIG. 2C. As described above, the implant parts 400 in the hip joint replacement surgery are formed by four parts which are each called an acetabular cup 402, a liner 404, a thigh bonehead 406 and a stem 408 as depicted in FIG. 2C. The polygon parts inserting section 14 holds, in advance, data of part objects of 3D shapes of these implant parts 400 modeled by means of 3D polygons, etc. Then, the polygon parts inserting section 14 puts these part objects to desired positions in the CT object 3D image, so as to produce a CT object 3D image after parts insertion. Incidentally, the terms to “put” the part objects in the CT object 3D image and to “insert” the part objects into the CT object 3D image are used for the same meaning.

Sizes of or where to put the part objects can be decided by the use of known arts disclosed in Japanese Unexamined Patent Publication No. 2006-263241, etc. Alternatively, the part objects may be arranged in size or in position alignment relative to the CT object 3D image by a manual operation using a mouse, etc.

It is practical as well to process the CT object 3D image in advance, before putting the part objects therein, so that the acetabular roof portion of the pelvis and the femoral head portion are trimmed in accordance with the acetabular cup 402 and the stem 408 in size in postoperative conditions, and then to put the part objects therein. In another case where the left and right thigh bones are different in lengths from the beginning, it is practical as well to align the position of the thigh bone requiring surgery by shifting the position (relative to the pelvis) in time of the insertion of the part objects.

In the thigh bone object before the insertion of the part objects, the center of the femoral head is the center of rotation of the hip joint. After the insertion of the part objects, on the other hand, the center of the thigh bone head 406 is, among the inserted part objects, the center of rotation of the hip joint. The polygon parts inserting section 14 searches for 3D coordinates of these rotation centers, and holds them as reference points to be used for position alignment with a intra-operative X-ray image described later (each called a “CT image reference point (before parts insertion)” and a “CT image reference point (after parts insertion)”, hereafter).

Incidentally, the ones of the part objects aligned in position corresponding to the stem 408 and the thigh bone head 406 are those fixed to the thigh bone object, and the ones corresponding to the acetabular cup 402 and the liner 404 are those fixed to the pelvis object. Suppose that a CT object 3D image into which the part objects are inserted is called a “CT object 3D image (after parts insertion)”. The CT object 3D image (before parts insertion) and the CT object 3D image (after parts insertion) are both made in the phase of the preoperative plan before the surgery.

The area including the patient's pelvis and the thigh bone is photographed by an X-ray system 300 intra-operatively with suitable timing. An image photographed by the X-ray system 300 during the surgery, i.e., an intra-operative X-ray image is a 2D image. The intra-operative X-ray image is stored in the X-ray image storing section 20 depicted in FIG. 2. X-ray images are photographed intra-operatively more than once, and are photographed before the implant parts are inserted, and after the implant parts are inserted.

The object position aligning section 30 aligns positions of the bone objects in the CT object 3D image (before parts insertion) made in the preoperative plan with positions of bone parts photographed in the intra-operative X-ray image. Alternatively, it aligns positions of the bone objects and the part objects in the CT object 3D image (after parts insertion) with positions of the bone parts and the implant parts in the intra-operative X-ray image.

The X-ray image bone outline extracting section 36 in the object position aligning section 30 extracts outlines of the bone parts (the pelvis and the left and right thigh bones) and the implant parts photographed in the intra-operative X-ray image, and produces a 2D intra-operative X-ray outline image.

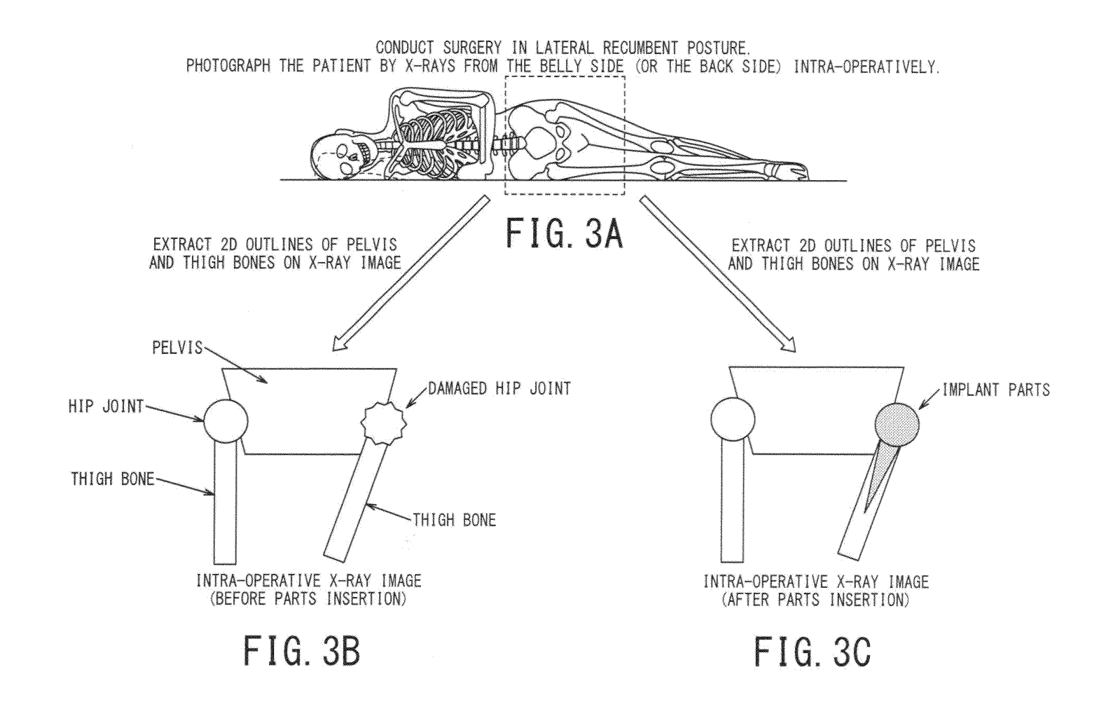

FIGS. 3A to 3C schematically illustrate the intra-operative X-ray outline image. As described above, the hip joint replacement surgery is conducted for a patient being in a lateral recumbent posture as depicted in FIG. 3A. Thus, the thigh bone on the side requiring surgery is after having rotated downwards around the hip joint.

Meanwhile, the patient being in the lateral recumbent posture is intra-operatively photographed by X-rays either from the patient's belly side or back side. Thus, the thigh bone on the side requiring surgery is after having rotated downwards around the hip joint in the intra-operative X-ray outline image as well, as depicted in FIGS. 3B and 3C. Incidentally, FIG. 3B exemplarily depicts an intra-operative X-ray outline image before the implant parts are inserted (called an “intra-operative X-ray outline image (before parts insertion)”, hereafter), and FIG. 3C exemplarily depicts an intra-operative X-ray outline image after the implant parts are inserted (called an “intra-operative X-ray outline image (after parts insertion)”, hereafter).

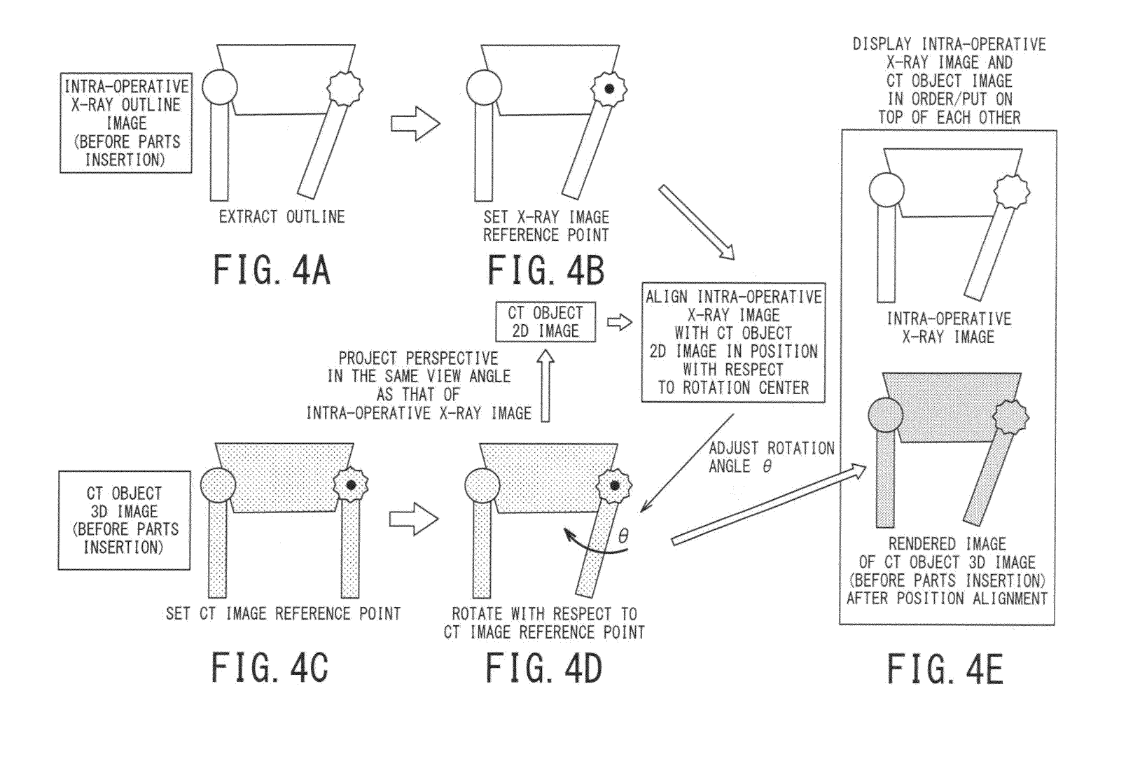

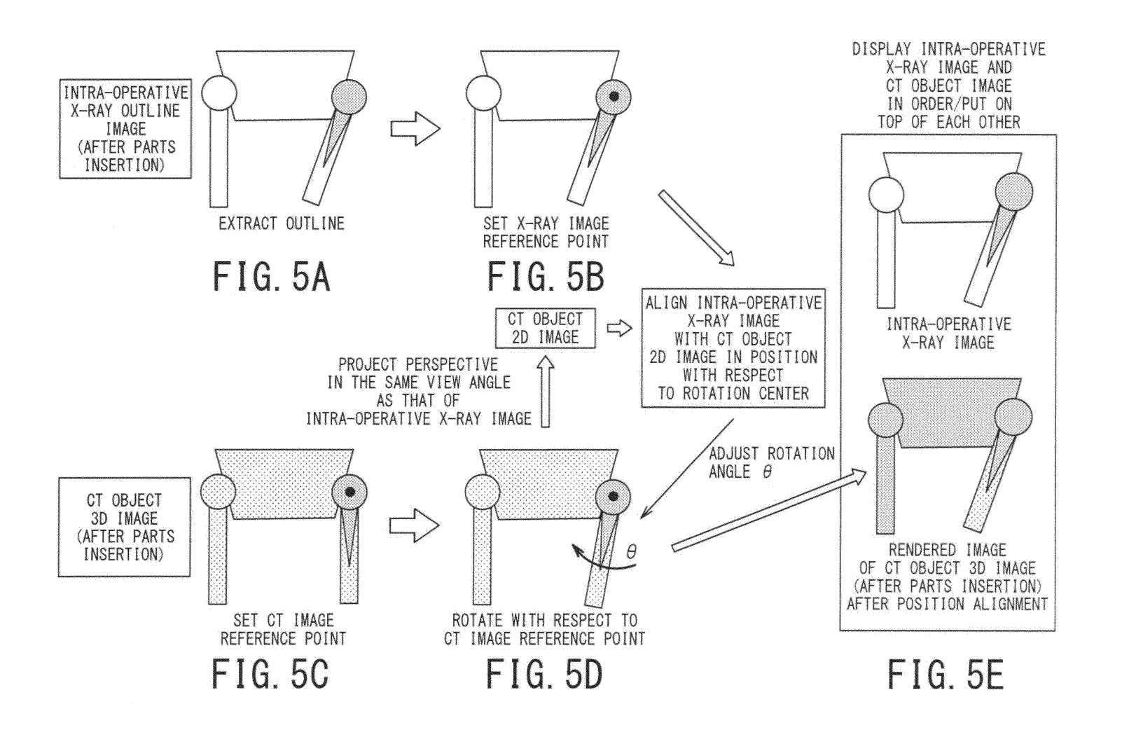

FIGS. 4A to 4E schematically illustrate data processing to align the position of the CT object 3D image (before parts insertion) with the position of the intra-operative X-ray outline image (before parts insertion). Similarly, FIGS. 5A to 5E schematically illustrate data processing to align the position of the CT object 3D image (after parts insertion) with the position of the intra-operative X-ray outline image (after parts insertion).

If the positions of the CT object 3D image (before parts insertion) and the intra-operative X-ray outline image (before parts insertion) are to be aligned with each other, the X-ray image position alignment reference point specifying section 37 in the object position aligning section 30 detects a position corresponding to the center of the femoral head on the basis of the outline shape of the thigh bone extracted in the intra-operative X-ray outline image (before parts insertion) (FIG. 4A), and renders the detected position an “X-ray image reference point (before parts insertion)” (a black plot in FIG. 4B). Meanwhile, if the positions of the CT object 3D image (after parts insertion) and the intra-operative X-ray outline image (after parts insertion) are to be aligned with each other, it detects a position corresponding to the center of the thigh bone head in the implant parts extracted in the intra-operative X-ray outline image (after parts insertion) (FIG. 5A) on the basis of the outline shape and the relative position, and renders the detected position an “X-ray image reference point (after parts insertion)” (a black plot in FIG. 5B).

On the other hand, the CT image reference point (before parts insertion) (a black plot in FIG. 4C) and the CT image reference point (after parts insertion) (a black plot in FIG. 5C) are each plotted in the CT object 3D image (before parts insertion) described above.

Thus, for the CT object 3D image (before parts insertion), the object rotating section 32 in the object position aligning section 30 rotates the thigh bone object around the CT image reference point (before parts insertion) by an unspecified angle θ in the beginning phase (FIG. 4D). Similarly, for the CT object 3D image (after parts insertion), it rotates the thigh bone object and the part objects (the stem and the thigh bone head) fixed thereto around the CT image reference point (after parts insertion) by the unspecified angle θ (FIG. 5D).

Then, the object outline projected image producing section 34 produces an image in which the CT object 3D image (before parts insertion) or the CT object 3D image (after parts insertion) having been rotated by the unspecified angle θ is projected in perspective along the same line of sight and in the same view angle as those of the intra-operative X-ray image (called a CT object 2D image (before parts insertion) or a CT object 2D image (after parts insertion), hereafter).

Before the insertion of the implant parts, then, the agreement deciding section 38 aligns the position of the CT object 2D image (before parts insertion) with the position of the intra-operative X-ray outline image (before parts insertion) in such a way that the CT image reference point (before parts insertion) in the CT object 2D image (before parts insertion) agrees with the X-ray image reference point (before parts insertion), and that the outline of the pelvis object in the CT object 2D image (before parts insertion) agrees with the outline of the pelvis in the intra-operative X-ray outline image (before parts insertion).

After the insertion of the implant parts, similarly, align the position of the CT object 2D image (after parts insertion) with the position of the intra-operative X-ray outline image (after parts insertion) in such a way that the CT image reference point (after parts insertion) in the CT object 2D image (after parts insertion) agrees with the X-ray image reference point (after parts insertion), and that the outline of the pelvis object in the CT object 2D image (after parts insertion) agrees with the outline of the pelvis in the intra-operative X-ray outline image (after parts insertion).

Then, the agreement deciding section 38 calculates a Mutual Information of the outline information of each of the two 2D images. That is, before the insertion of the implant parts, calculate a Mutual Information of the outline information of the CT object 2D image (before parts insertion) and the intra-operative X-ray outline image (before parts insertion). After the insertion of the implant parts, calculate a Mutual Information of the outline information of the CT object 2D image (after parts insertion) and the intra-operative X-ray outline image (after parts insertion).

The Mutual Information mentioned here is a quantitative index which indicates how much two images correlate with each other. The Mutual Information can be calculated by the use of a method described, e.g., in a document “W R Crum, D L G Hill, D J Hawkes (2003) Information theoretic similarity measures in non-rigid registration, IPMI-2003, pp. 378-387”.

Then, the agreement deciding section 38 decides whether the calculated Mutual Information has converged on a sufficiently high value.

If the agreement deciding section 38 decides that the convergence is insufficient, return to the data processing run by the object rotating section 32. The object rotating section 32 further rotates the thigh bone object (or the thigh bone object and the part objects fixed thereto which are the stem and the thighbone head) by another unspecified angle θ, and the object outline projected image producing section 34 again produces a CT object 2D image (before parts insertion) or a CT object 2D image (after parts insertion). Then, the agreement deciding section 38 again decides agreement by using the Mutual Information.

The object rotating section 32, the object outline projected image producing section 34 and the agreement deciding section 38 align the position of the CT object, 3D image with the position of the intra-operative X-ray outline image while using rotation angels of the pelvis and the thigh bone as parameters according to a method of successive approximation by using the Mutual information in this way. Change the rotation angles of the pelvis and the thigh bone in a direction in which the Mutual Information rises, so that the processing of successive approximation can converge.

Although the outline data of two images are to be aligned in position with each other, it is also practical to extract the areas of the pelvis and the thigh bone (further, the area of the implant parts after parts insertion) of both the images, and to have area data of the objects aligned in position with each other. Further, it is also practical to have pixel data of both the images aligned in position with each other. In this case, the term “intra-operative X-ray outline image” described above can be replaced with “intra-operative X-ray bone part extracted image”.

If the agreement deciding section 38 decides a sufficient degree of convergence and if it is decided that an angle between the pelvis and the thigh bone in the CT object 3D image has sufficiently agreed with an angle between the pelvis and the thigh bone in the intra-operative X-ray outline image, the object rotating section 32 provides the image synthesizing section 40 with the CT object 3D image (the CT object 3D image aligned in position).

The image synthesizing section 40 produces an image as a reference image that the CT object 3D image aligned in position is rendered by means of a method such as surface polygon rendering. Then, before the parts insertion, display the rendered image (reference image) of the CT object 3D image (before parts insertion) and the intra-operative X-ray image (before parts insertion) in order as depicted in FIG. 4E. Alternatively, provide the display section 50 with the reference image and the intra-operative X-ray image (before parts insertion) put on top of each other. After the parts insertion, further, similarly display the rendered image (reference image) of the CT object 3D image (after parts insertion) and the intra-operative X-ray image (after parts insertion) in order in a column as depicted in FIG. 5E. Alternatively, provide the display section 50 with the reference image and the intra-operative X-ray image (after parts insertion) put on top of each other. The display section 50 displays these images on a display screen.

While the intra-operative X-ray image (after parts insertion) is of the positions of the practically inserted implant parts or the implant parts being practically inserted photographed, the CT object 3D image (after parts insertion) indicates the positions of the implant parts decided in the preoperative plan. Thus, the surgeon compares the two images with each other so that the surgeon can easily decide whether the implant parts are inserted into planned positions.

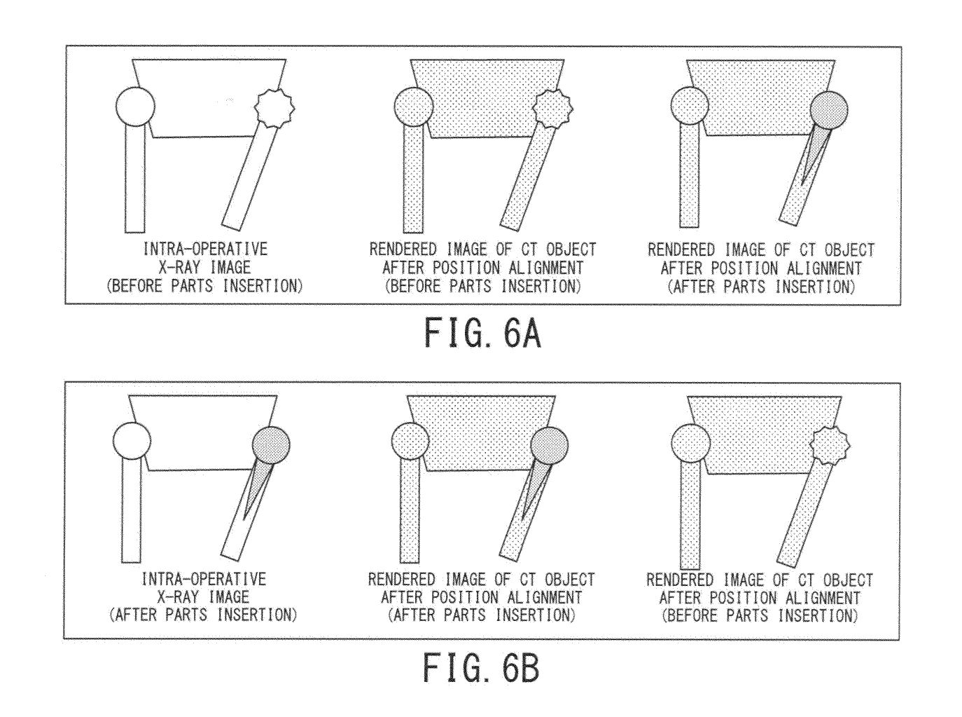

Types of the images for being displayed are not limited to the above, and various forms can be practical. Before the parts insertion, e.g., a rendered image of the CT object 3D image (after parts insertion) can be displayed in addition to the rendered image of the CT object 3D image (before parts insertion) and the intra-operative X-ray image (before parts insertion) displayed parallel to each other as depicted in FIG. 6A. Alternatively, after the parts insertion, e.g., a rendered image of the CT object 3D image (before parts insertion) can be displayed in addition to the rendered image of the CT object 3D image (after parts insertion) and the intra-operative X-ray image (after parts insertion) displayed parallel to each other as depicted in FIG. 6B.

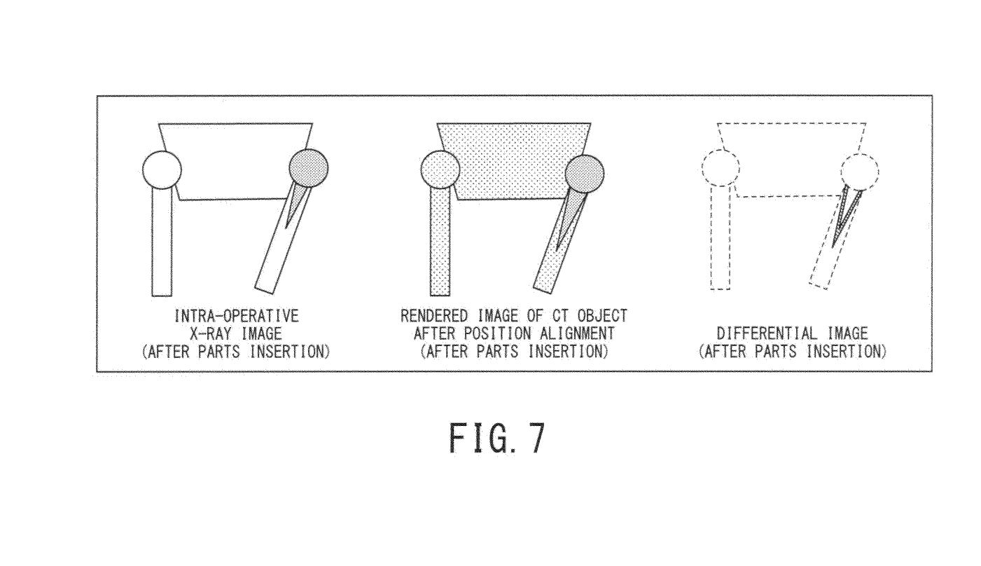

After the parts insertion, further, a differential image between the two images can be displayed in addition to the rendered image of the CT object 3D image (after parts insertion) and the intra-operative X-ray image (after parts insertion) displayed parallel to each other as depicted in FIG. 7. The differential image indicates differences between the positions of the implant parts practically inserted or being inserted and the positions of the implant parts decided in the preoperative plan more directly. Thus, the surgeon can immediately decide whether the implant parts are exactly inserted into the planned positions as intra-operatively monitoring how large the difference is.

Incidentally, it is also practical to intra-operatively photograph X-ray images from two and over directions, to provide a detector which can detect depths of the insertions of the implant parts inserted into the thigh bone more precisely, so as to compare the depths of the insertions of the implant parts in the CT object 3D image (after parts insertion) made in the preoperative plan with the depths of the implant parts detected from the intra-operative X-ray image more precisely.

### (2) Second Embodiment

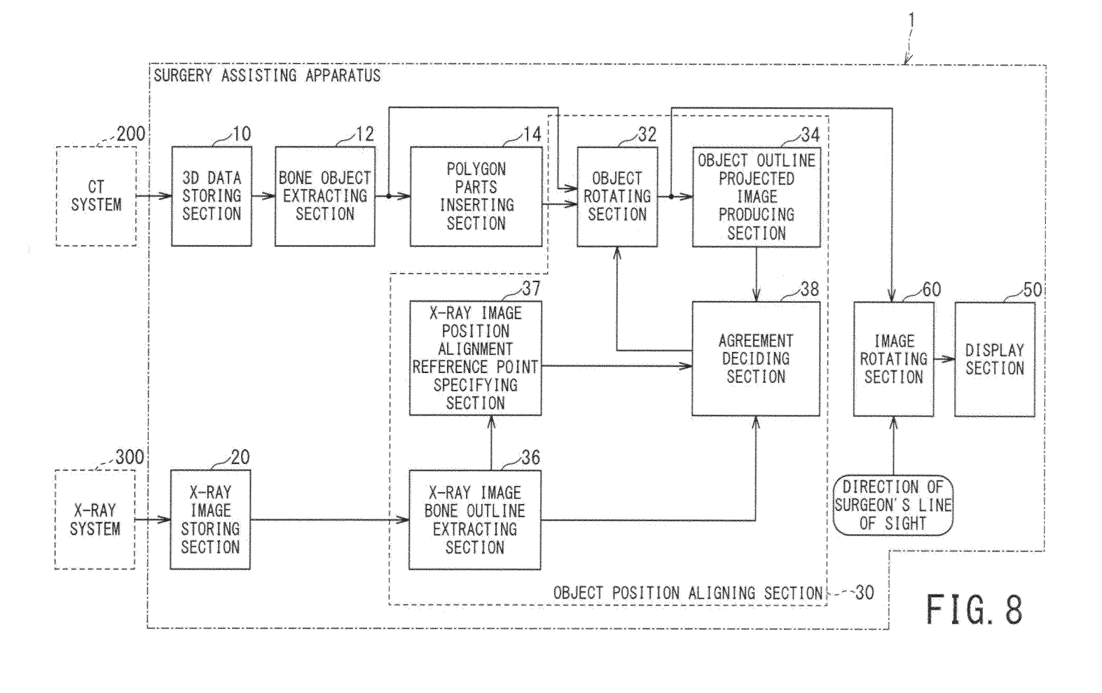

FIG. 8 depicts an exemplary setup of a surgery assisting apparatus 1 of a second embodiment. The surgery assisting apparatus 1 of the second embodiment is formed by having an image rotating section 60 which rotates and displays CT object 3D images having been aligned in position at an angle viewed along a surgeon's line of sight.

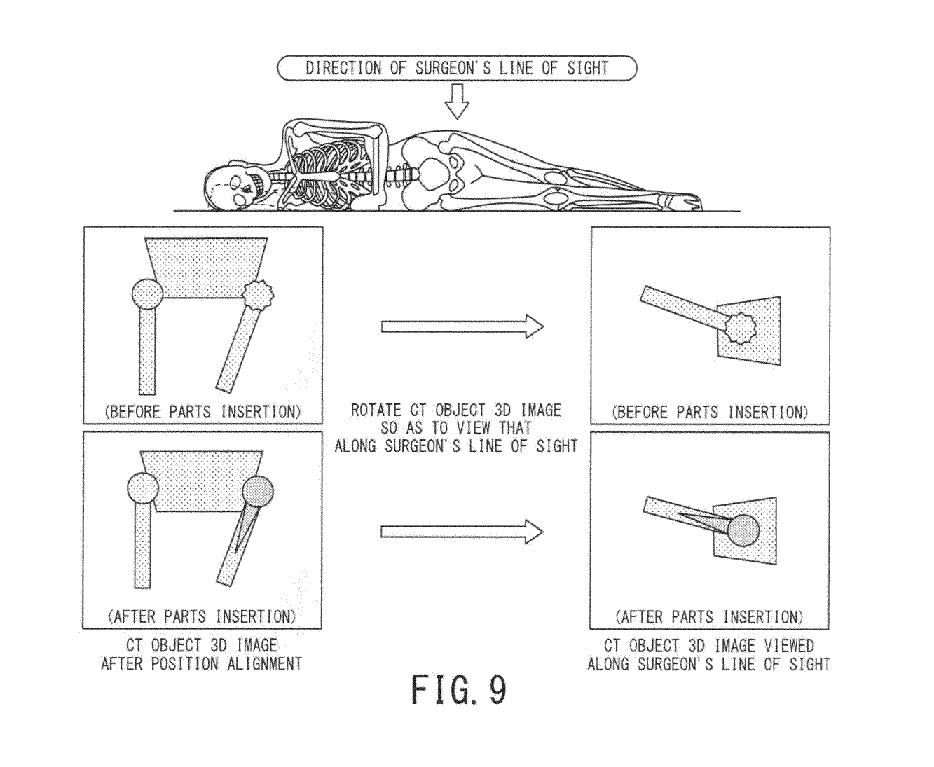

FIG. 9 schematically illustrates how the image rotating section 60 works. As described above, the hip joint replacement surgery is conducted for a patient being in a lateral recumbent posture, and a surgeon is supposed to conduct the surgery while usually looking down from above the patient at the hip joint part requiring surgery and the thigh bone. Thus, the image rotating section 60 of the surgery assisting apparatus 1 of the second embodiment rotates CT object 3D images having been aligned in position by the object position aligning section 30, and makes their directions agree with the direction of the surgeon's line of sight. For instance, rotate a CT object 3D image (before parts insertion) or a CT object 3D image (after parts insertion) having been aligned in position in such a way that the upper side of the screen agrees with the anterior side of the patient, that the lower side of the screen agrees with the posterior side of the patient, and that the thigh bone requiring surgery is on the front side of the screen, so as to produce an image rendered in a direction from the front of the screen. Then, display the rendered image on the display section 50 as a reference image.

According to the surgery assisting apparatus 1 of the second embodiment, the hip joint part in the CT object 3D image produced in the preoperative plan is aligned in position in such a way that it agrees with the intra-operative bending conditions of the patient's hip joint, and further the CT object 3D image aligned in position is displayed on the display section 50 as a rendered image viewed along the surgeon's line of sight. Thus, the surgeon can be provided with a more useful assisting image.

Incidentally, it is practical to display a rendered image of a CT object 3D image produced by the first embodiment viewed in the same direction as the direction of the intra-operative X-ray image and a rendered image of a CT object 3D image produced by the second embodiment viewed along the surgeon's line of sight parallel to each other at the same time, and to display them alternately one by one as well.

### (3) Third Embodiment

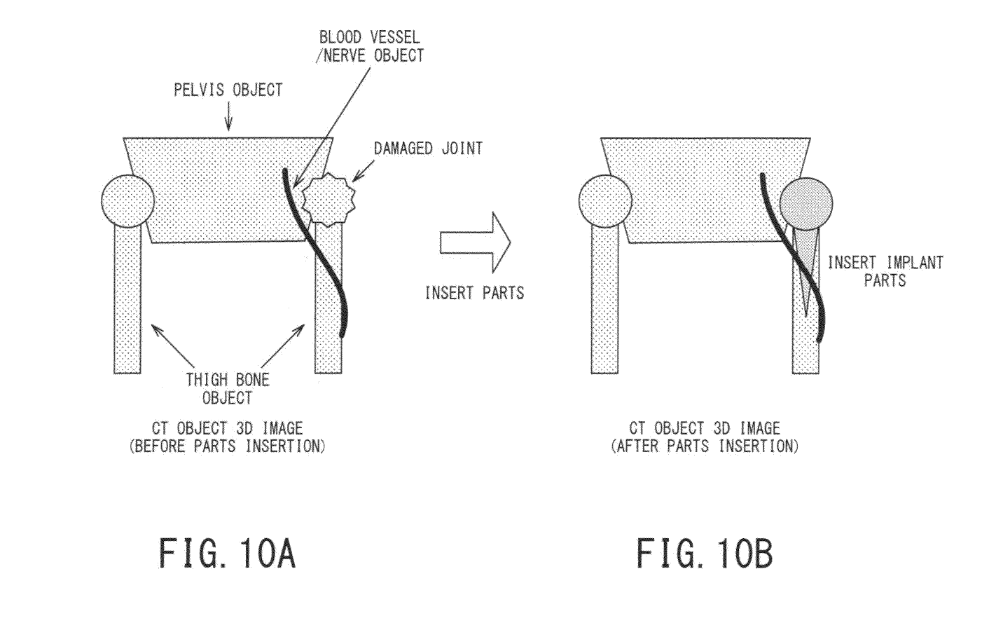

The bone object extracting section 12 of one of the first and second embodiments described above is supposed to extract 3D object data which corresponds to the pelvis, the right thigh bone and the left thigh bone, i.e., the bone objects from the 3D image data stored in the 3D data storing section 10. According to a third embodiment, on the other hand, extract a blood vessel or a nerve running close to the part requiring surgery as an object similarly as and in addition to the bone objects as depicted in FIG. 10A (the object which corresponds to a blood vessel or a nerve is called a blood vessel/nerve object, hereafter). That is, the CT object 3D image (before parts insertion) is a 3D image formed by including a bone object and a blood vessel/nerve object.

Then, the polygon parts inserting section 14 inserts part objects of the implant parts into the 3D image formed by including the bone object and the blood vessel/nerve object as depicted in FIG. 10B.

According to the third embodiment, a CT object 3D image (before parts insertion) and a CT object 3D image (after parts insertion) which each include a blood vessel/nerve object are produced in this way.

The object position aligning section 30 runs the same data processing as that of the first and second embodiments, and the angle θ between the pelvis object and the thigh bone object in the CT object 3D image (before parts insertion), and in the CT object 3D image (after parts insertion) as well, is determined in such a way as to agree with the angle between the pelvis and the thighbone in the intra-operative X-ray image.

When rotating the thighbone object around the CT image reference point, deform, move and rotate the blood vessel/nerve object while keeping its position relative to the pelvis object and the thigh bone object. For deformation, further, bend a blood vessel (or nerve) existing on the pelvis side and a blood vessel (or nerve) existing on the thigh bone side on the basis of a rotation angle between the pelvis and the thigh bone.

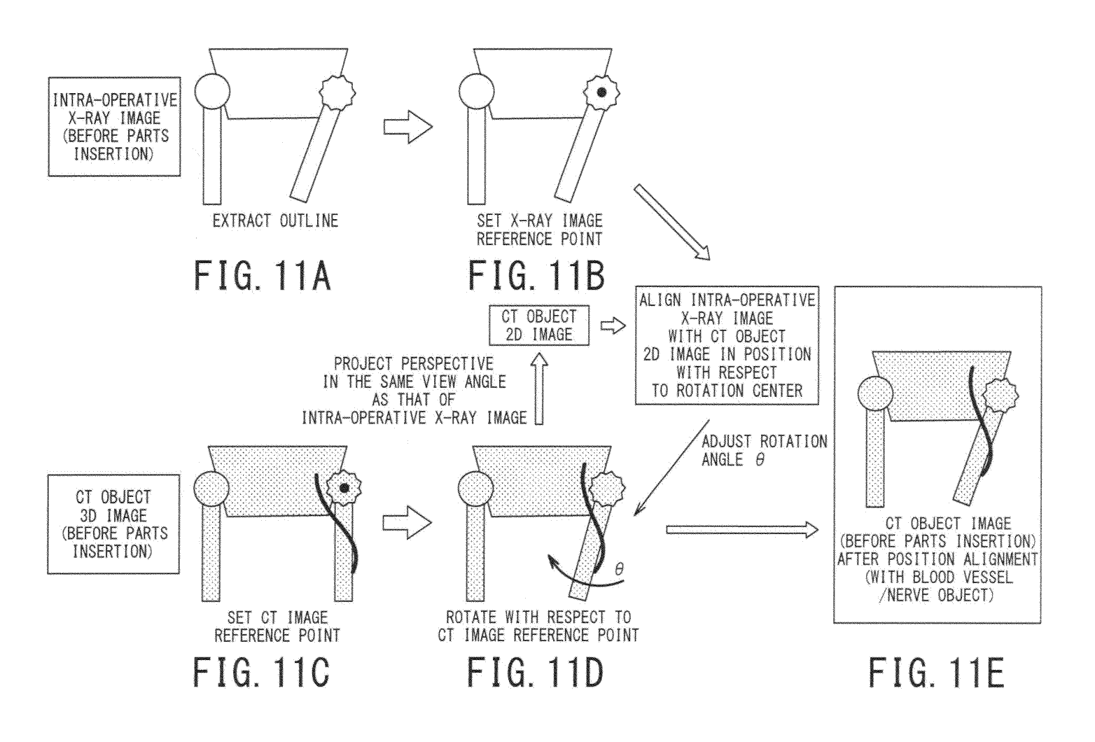

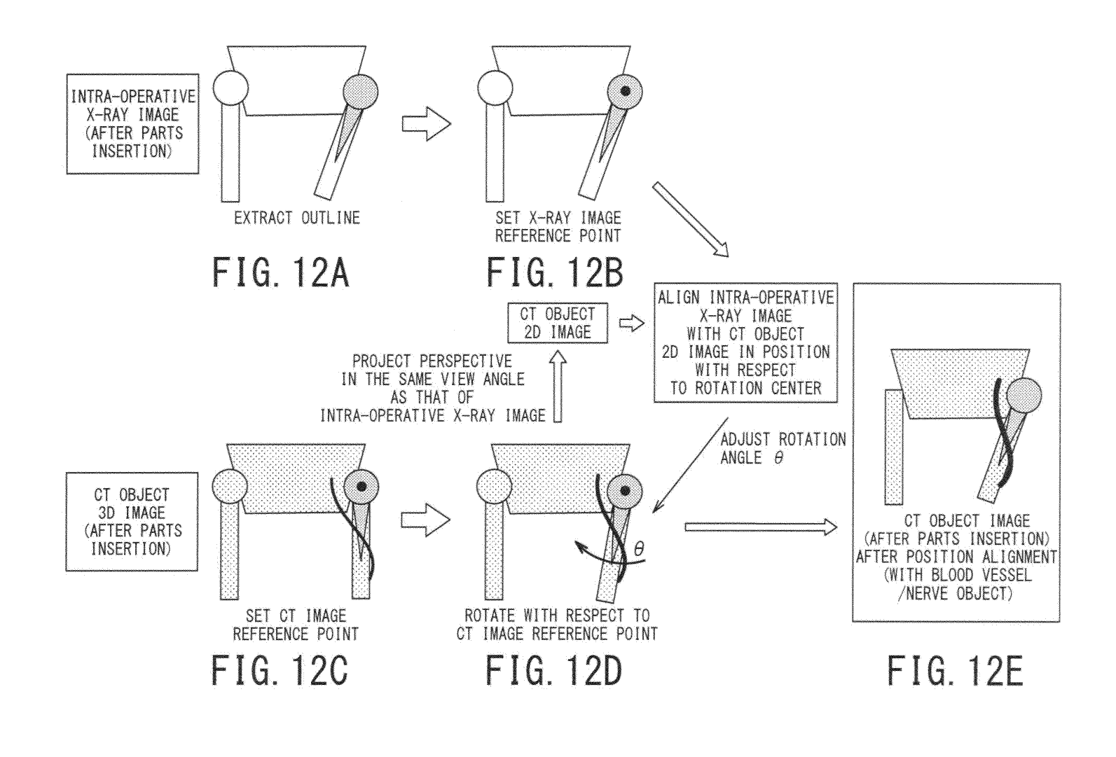

FIGS. 11 and 12 each schematically illustrate position alignment in a CT object 3D image (before parts insertion) and in a CT object 3D image (after parts insertion) each with a blood vessel/nerve object using an intra-operative X-ray image, respectively. According to the third embodiment, rendered images of a CT object 3D image (before parts insertion) and a CT object 3D image (after parts insertion) are supposed to be displayed on the display section 50 as reference images. Incidentally, as what is substantially processed is more or less the same as depicted in FIGS. 4 and 5, detailed explanation is omitted.

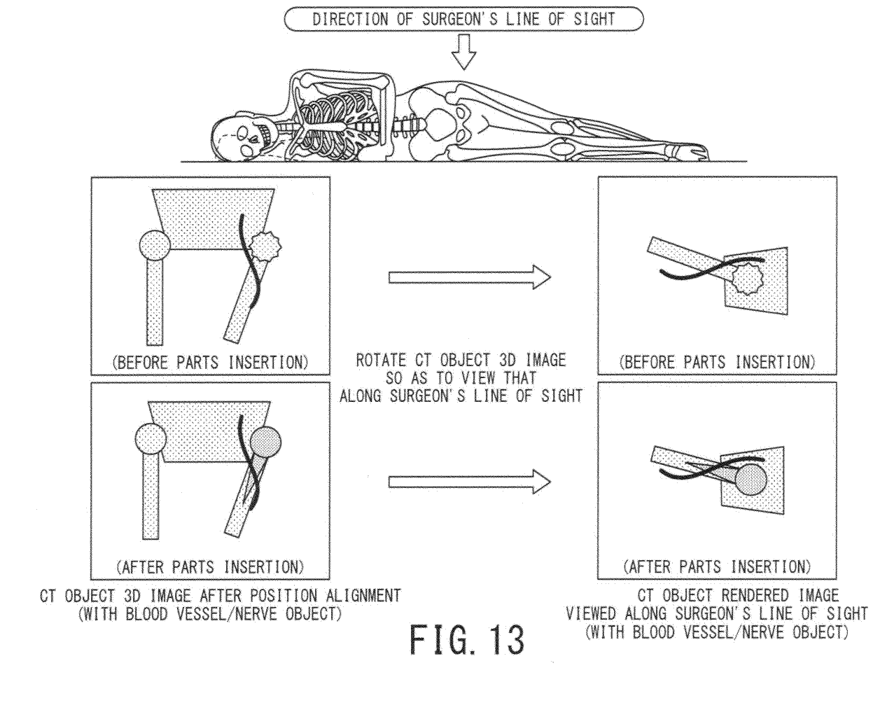

Further, the third embodiment may be combined with the second embodiment as depicted in FIG. 13. That is, rotate the CT object 3D image with a blood vessel/nerve object having been aligned in position, and display a rendered image viewed along the surgeon's line of sight on the display section 50 as a reference image.

As previously described, it becomes more frequent in recent years to conduct a minimum invasive surgery (MIS), i.e., a surgery by invading through an extremely small resection area from a viewpoint of reducing a burden on the patient, etc. As the resection area is small in this type of the minimum invasive surgery, there are problems in that it is hardly known intra-operatively how the thigh bone and the implant parts are relatively located to one another, that it is hardly known whether the implant parts are inserted into the right position in the right angle, and that it is hardly grasped intra-operatively how the blood vessels and the nerves not to be damaged run as the resection area is small.

According to the respective embodiments described above, insertion conditions of the implant parts in the phase of surgery planning can be easily compared with insertion conditions of the implant parts in an intra-operatively obtained X-ray photographed image. Further, the surgeon can easily compare intra-operative insertion conditions of the implant parts and surrounding blood vessels and nerves viewed from a small resection area. Further, presence or position of a blood vessel or a nerve being placed at a position that the surgeon can hardly look at can be easily grasped by means of reference to a CT object 3D image with a blood vessel/nerve object.

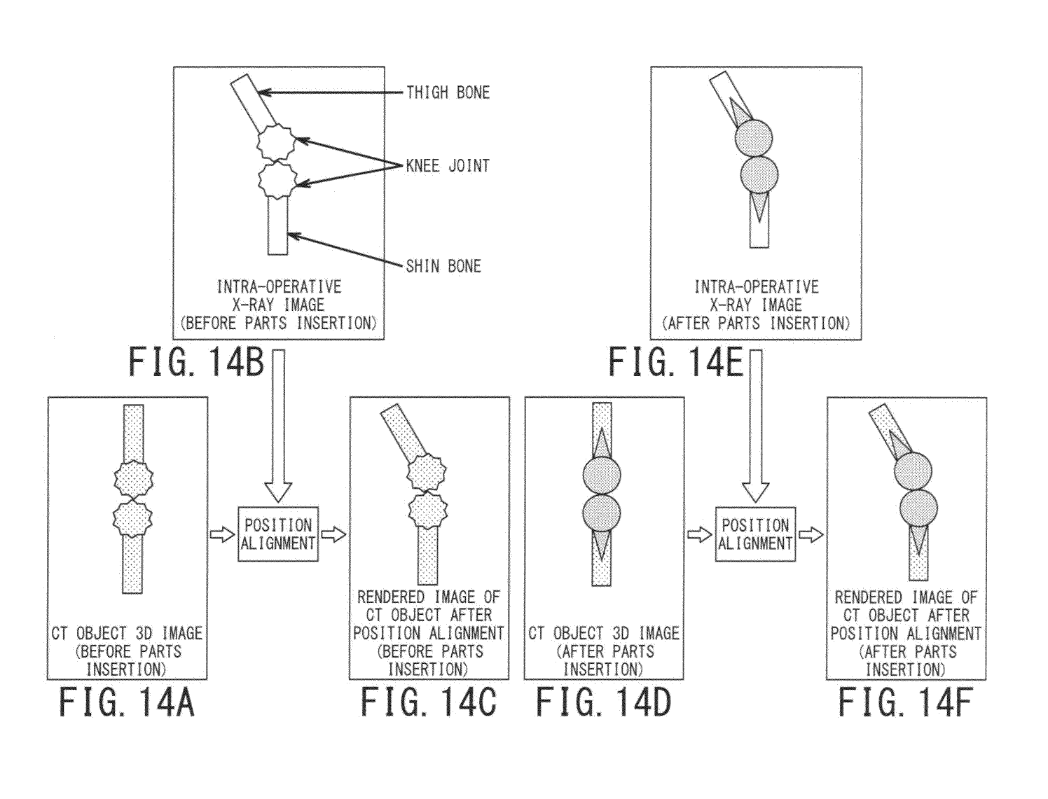

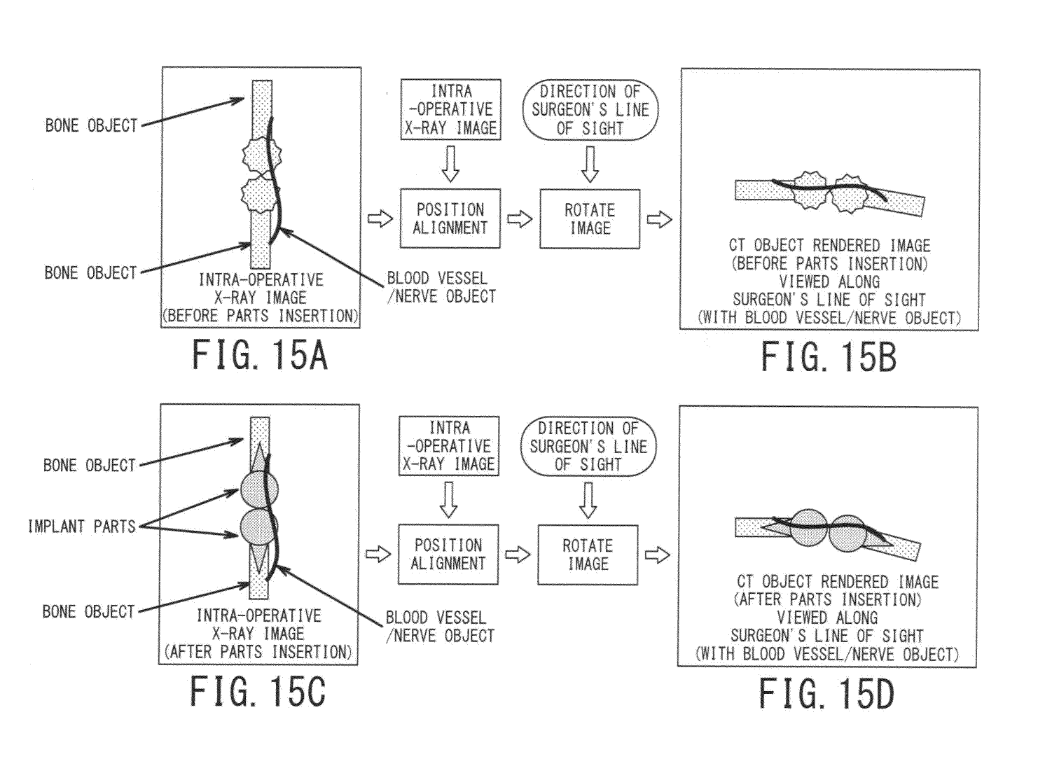

The above description has explained the respective embodiments of the surgery assisting apparatus 1 by taking an example of a hip joint replacement surgery, and the surgery assisting apparatus 1 can be applied to a joint replacement surgery excepting the hip joint replacement surgery as a matter of course. FIGS. 14 and 15 each illustrate an exemplary application of the surgery assisting apparatus 1 to an artificial knee joint replacement surgery.

In an artificial knee joint replacement surgery, the knee joint requiring surgery and the thigh bone (first bone part) and the shin bone (second bone part) on the both sides are photographed in a 3D CT image from which a CT object 3D image (before parts insertion) in which the respective bone objects are extracted is produced as depicted in FIG. 14A, similarly as in a hip joint replacement surgery. Then, the CT object 3D image (before parts insertion) is aligned in position in such a way as to agree with bending of the knee part which appears in an intra-operatively (before implant parts insertion) photographed intra-operative X-ray image (FIG. 14B) (FIG. 14C). Further, a CT object 3D image (after parts insertion) in which part objects are inserted into the CT object 3D image (before parts insertion) is produced as a preoperative plan (FIG. 14D), and the CT object 3D image (after parts insertion) is similarly aligned in position in such a way as to agree with bending of the knee part which appears in an intra-operatively (after implant parts insertion) photographed intra-operative X-ray image (FIG. 14E) (FIG. 14F). Then, a rendered image of the CT object 3D image aligned in position (reference image) and the intra-operative X-ray image are displayed on the display section 50 being put in order or on top of each other.

As depicted in FIGS. 15A and 15C, further, it is also practical to extract a blood vessel or a nerve existing around the knee joint as a blood vessel/nerve object in addition to the bone object of the thigh bone or the shin bone, to produce a CT object 3D image (before parts insertion) or a CT object 3D image (after parts insertion) with a blood vessel/nerve object, to align that in position with the intra-operative X-ray image and then display that on the display section 50 as a reference image.

As depicted in FIGS. 15B and 15D, still further, it is also practical to rotate a CT object 3D image (before parts insertion) or a CT object 3D image (after parts insertion) with a blood vessel/nerve object aligned in position in a direction along the surgeon's line of sight, to do rendering on it and to display the rendered image on the display section 50 as a reference image.

The embodiments of the invention having been explained are presented as exemplary only, and it is not intended to limit the scope of the invention. These embodiments can be practiced in other various forms, and can be variously omitted, replaced or changed within the gist of the invention. The inventions and their modifications are included in the scope and the gist of the invention, and in the inventions described in the claims and their equivalents as well.

1. A surgery assisting apparatus configured to assist a surgery to replace a joint of a patient with an artificial joint, the surgery assisting apparatus comprising:
a bone object extracting section configured to produce a 3D object image in which a first bone object and a second bone object are each separated and extracted from a 3D image in which a diseased part including the joint, a first bone part and a second bone part movably connected with the first bone part via the joint are photographed, the first bone object and the second bone object corresponding to the first bone part and the second bone part, respectively;
an object position aligning section configured to input an X-ray image in which the diseased part is photographed in the course of the surgery of the patient, the object position aligning section being configured, while extracting the first bone part and the second bone part in the inputted X-ray image and producing an intra-operative X-ray bone part extracted image, to align the 3D object image in position in such a way that the first bone object and the second bone object agree with the first bone part and the second bone part in the intra-operative X-ray bone part extracted image, respectively, so as to produce a reference image; and
a display section on which the X-ray image and the reference image are displayed.

2. The surgery assisting apparatus according to claim 1, wherein the object position aligning section is configured:
while searching for a rotation center of the joint in the intra-operative X-ray bone part extracted image from the first bone part and the second bone part extracted from the X-ray image;
to search for a rotation center of the joint in the 3D object image from the first bone object and the second bone object;
to make a position of the rotation center of the joint in the 3D object image agree with a position of the rotation center of the joint in the intra-operative X-ray bone part extracted image; and
to align the first bone object and the second bone object with the first bone part and the second bone part in position, respectively, in the intra-operative X-ray bone part extracted image by rotating one of the first bone object and the second bone object with respect to the rotation center of the joint in the 3D object image, so as to produce the reference image.

3. The surgery assisting apparatus according to claim 1, wherein:
the 3D object image is an image of the patient photographed in a supine posture;
the X-ray image is an image of the patient being photographed intra-operatively in a lateral recumbent posture; and
the reference image is produced in such a way that a direction of projection or rendering on the 3D object image agrees with a direction in which the X-ray image is photographed.

4. The surgery assisting apparatus according to claim 1, wherein the 3D object image is an image after the artificial joint is replaced according to a preoperative plan, and the reference image is an image produced from the 3D object image after the artificial joint replacement.

5. The surgery assisting apparatus according to claim 1, wherein the 3D object image is an image before the artificial joint is replaced, and the reference image is an image produced from the 3D object image before the artificial joint replacement.

6. The surgery assisting apparatus according to claim 3, wherein:
the artificial joint has implant parts to be inserted into the second bone part; and
the X-ray image is an image photographed in such a way that a depth of insertion of the implant parts can be detected.

7. The surgery assisting apparatus according to claim 1, wherein:
the 3D object image is an image of the patient photographed in a supine posture;
the X-ray image is an image of the patient being photographed intra-operatively in a lateral recumbent posture;
the object position aligning section further produces a second reference image which agrees with a direction of a surgeon's line of sight onto the patient from the 3D object image; and
the second reference image is further displayed on the display section.

8. The surgery assisting apparatus according to claim 7, wherein the 3D object image is an image after the artificial joint is replaced according to a preoperative plan, and the second reference image is an image produced from the 3D object image after the artificial joint replacement.

9. The surgery assisting apparatus according to claim 7, wherein the 3D object image is an image before the artificial joint is replaced, and the second reference image is an image produced from the 3D object image before the artificial joint replacement.

10. The surgery assisting apparatus according to claim 7, wherein:
the bone object extracting section produces from the 3D image an image in which at least one of a blood vessel and a nerve existing close to the diseased part is separated and extracted as a blood vessel/nerve object in addition to the first and second bone objects as the 3D object image; and
the object position aligning section, while maintaining positions of the first bone object and the second bone object relative to the blood vessel/nerve object, aligns the first bone object and the second bone object in position in such a way that the first bone object and the second bone object agree with the first bone part and the second bone part, respectively, in the X-ray bone part extracted image, and produces a reference image which agrees with a direction of a surgeon's line of sight and includes the blood vessel/nerve object as the second reference image.

11. The surgery assisting apparatus according to claim 10, wherein the 3D object image is an image after the artificial joint is replaced according to a preoperative plan, and the second reference image is an image produced from the 3D object image after the artificial joint replacement.

12. The surgery assisting apparatus according to claim 10, wherein the 3D object image is an image before the artificial joint is replaced, and the second reference image is an image produced from the 3D object image before the artificial joint replacement.

13. The surgery assisting apparatus according to claim 1, wherein the first bone part, the second bone part and the joint are a pelvis, a thigh bone and a hip joint, respectively.

14. The surgery assisting apparatus according to claim 1, wherein the first bone part, the second bone part and the joint are a thigh bone, a shin bone and a knee joint, respectively.

15. The surgery assisting apparatus according to claim 2, wherein the first bone part, the second bone part and the joint are a pelvis, a thigh bone and a hip joint, respectively.

16. The surgery assisting apparatus according to claim 3, wherein the first bone part, the second bone part and the joint are a pelvis, a thigh bone and a hip joint, respectively.

17. The surgery assisting apparatus according to claim 4, wherein the first bone part, the second bone part and the joint are a pelvis, a thigh bone and a hip joint, respectively.

18. The surgery assisting apparatus according to claim 2, wherein the first bone part, the second bone part and the joint are a thigh bone, a shin bone and a knee joint, respectively.

19. The surgery assisting apparatus according to claim 3, wherein the first bone part, the second bone part and the joint are a thigh bone, a shin bone and a knee joint, respectively.

20. The surgery assisting apparatus according to claim 4, wherein the first bone part, the second bone part and the joint are a thigh bone, a shin bone and a knee joint, respectively.
